(19) United States
(12) Patent Application Publication
(10) Pub. No.: US 2026/0272998 A1
(43) Pub. Date: Sep. 17, 2026

(54) CHEMICALLY-MODIFIED MICROORGANISM CONTAINING A SUPEROXIDE DISMUTASE MIMIC INORGANIC COMPLEX AND USE THEREOF FOR TREATING INFLAMMATORY DISEASES

(71) Applicants: INSTITUT NATIONAL DE RECHERCHE POUR L'AGRICULTURE, L'ALIMENTATION ET L'ENVIRONNEMENT, Paris (FR); INSTITUT NATIONAL DES SCIENCES ET INDUSTRIES DU VIVANT ET DE L'ENVIRONNEMENT, Palaiseau (FR); ÉCOLE NORMALE SUPÉRIEURE, Paris (FR); ASSISTANCE PUBLIQUE-HÔPITAUX DE PARIS, Paris (FR); INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); SORBONNE UNIVERSITE, Paris (FR)

(72) Inventors: Anne AUCOUTURIER, Vélizy (FR); Luis BERMUDEZ, Saclay (FR); Florian CHAIN, Versailles (FR); Nicolas DELSUC, Paris (FR); Philippe LANGELLA, Vélizy (FR); Clotilde POLICAR, Paris (FR); Elodie QUEVRAIN, Bois le Roi (FR); Gabrielle SCHANNE, Ivry-sur-Seine (FR); Philippe SEKSIK, Vincennes (FR); Amandine VINCENT, Poitiers (FR)

(21) Appl. No.: 19/470,885

(22) PCT Filed: Mar. 28, 2024
(86) PCT No.: PCT/EP2024/058658

§ 371 Date: Sep. 30, 2025

(30) Foreign Application Priority Data
Mar. 31, 2023 (EP) 23305463.4

(51) Int. Cl. A61K 35/744; A61K 31/555; A61P 1/00; A61P 29/00; C12N 9/02

(52) U.S. Cl. CPC A61K 35/744; A61K 31/555; A61P 1/00; A61P 29/00; C12N 9/0089; C12Y 115/01001

(57) ABSTRACT

A chemically-modified microorganism selected from bacteria and yeasts, which contains a superoxide dismutase mimic inorganic complex, in particular a mimic of human manganese superoxide dismutase. This microorganism is particularly useful for treating an inflammatory disease, in particular an inflammatory bowel disease, by oral administration.

The invention lies in the field of drug delivery, in particular of intestinal delivery of antioxidant drugs intended for combating oxidative stress.

More particularly, the invention relates to the use as a medicament or a dietary supplement, in particular for combating oxidative stress, and more particularly for treating inflammatory diseases, of a chemically-modified microorganism containing a Superoxide Dismutase (SOD) mimic. The invention also relates to a pharmaceutical composition, as well as a nutraceutical composition, food composition or dietary supplement, containing such a microorganism.

Superoxide Dismutases (SODs) are metalloenzymes involved in the cellular antioxidant defenses. In the organism SODs are responsible for maintaining superoxide, a reactive oxygen species (ROS), at tightly controlled levels and contribute to prevent oxidative stress situations known to be implicated in various diseases. In their active site, all the SODs contain a metal cation that cycles between two redox states to successively reduce superoxide to H₂O₂ and to oxidize it into O₂. In humans, three different kinds of eukaryotic SODs have been described, depending on the metal contained in their active site: two copper/zinc SODs, SOD1 found in the cytosol and in the mitochondrial intermembrane space and SOD3 found in the extracellular environment, within the extracellular matrix and at cell surface, and the manganese SOD (SOD2), also called MnSOD, localized in the mitochondria matrix (Weisiger and Fridovich, 1973, Journal of Biological Chemistry. 248:4793-4796).

Inflammatory Bowel Diseases (IBDs) are chronic disorders characterized by a chronical inflammation of part of the gastro-intestinal tract. They include Crohn's disease and ulcerative colitis, that differ at least by the location of the inflammation. Both can cause diarrhea, rectal bleeding, anemia, weight loss and abdominal pains. IBDs constitute a global health issue as their incidence is particularly high, and keeps increasing, in developed countries, reaching in cumulative incidence, over life time, one percent in Europe. The number of IBDs cases in the world is estimated at 6.8 million people. For now, no curative therapy exists to treat IBDs and patients mostly receive drugs aiming to control inflammation in order to alleviate the symptoms and to prevent the flare-ups. The main treatments include corticosteroids and immunomodulators comprising small molecules and monoclonal antibodies targeting specific inflammatory pathways or cytokines (Paramsothy et al., 2018, Mucosal Immunol. 11:1558-1570). However, these expensive drugs are not always efficient and can induce loss of response and adverse effects. There is therefore an urgent need for novel, safe and efficient therapeutic alternatives to fight IBDs.

Inflammatory bowel diseases have been described to be accompanied by an over-expression of the intestinal MnSOD in an enzymatically inactive form and by an under-expression of the intestinal cytoplasmic Cu/Zn SOD (Kruidenier et al., 2003, J. Pathol. 201:7-16). These deficiencies in the SOD antioxidant system may cause or, at least, exacerbate the oxidative stress observed in IBDs which is known to be closely related to chronic inflammation (Krzystek-Korpacka et al., 2020, Diagnostics. 10:601). A SOD-based antioxidant treatment has thus appeared as a promising therapy for IBDs. MnSODs in particular were shown to efficiently reduce lipid peroxidation and neutrophil recruitments and to attenuate the inflammation in both DSS-induced and TNBS-induced colitis murine models (Naito et al., 2005, CDTIA. 4:511-515). WO 2020/050460 discloses a composition for treating IBDs, comprising a mutant strain of the Bacillus amyloliquefaciens bacteria with a high rate of production of the SOD enzyme, and/or the SOD enzyme purified from such a strain after incubation of the cells with a manganese salt. However, the use of these purified enzymes as therapeutics is limited by their short half-life, the triggered immunogenicity and their low cell-penetration.

To overcome these shortcomings, synthetic low-molecular weight SOD mimics, also called SOD mimetics, mimicking SOD activity, i.e., capable of catalyzing superoxide dismutation, were examined as therapeutic candidates for IBDs management. A large variety of synthetic SOD mimic antioxidant inorganic complexes have been reported, including iron, copper, zinc and manganese complexes. These synthetic inorganic complexes generally have a molecular weight of less than 10 kDa, and often less than 5 kDa. Manganese complexes, in particular, appeared to be more favorable in comparison with Cu, Fe and Ni complexes since manganese, if released, is better tolerated by cells and does not catalyze the Fenton or Haber-Weiss reactions that lead to the formation of the extremely reactive and toxic HO· radical.

Many manganese-based SOD mimics have been described in the literature, including for example complexes with ligands such as salen derivatives, cyclic polyamine, 1,2-ethane-diamine-centered ligands, porphyrins, phthalocyanines, etc. (Vincent et al., 2021, Journal of Inorganic Biochemistry 219:111431).

As an example, a manganese complex mimicking SOD, called Mn1, of formula (V):

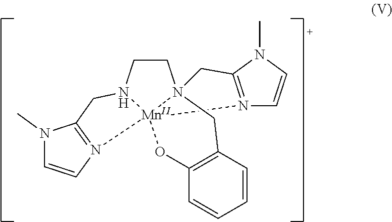

has been reported to exert an intracellular anti-inflammatory activity in vivo in a DBNS-induced colitis murine model, demonstrated by analysis of the weight variation and macroscopic scores, when administered orally in a carbonate buffer (Mathieu et al., 2017, Inorg. Chem., 56:2545-2555). This activity was however limited, probably due to an instability of this inorganic complex in the biological environment. Additionally, although the use of a basic buffer as a vehicle for the metal complex improves the stability thereof in acid environments, such a vehicle is not desirable for administration to humans.

The publication of Karlsson et al., 2015, Drug Discovery Today, 20:411-421, discloses the intracellular intake of an SOD mimic, Mn Salen, in MnSOD-deficient Cryptococcus neoformans.

The present invention aims to propose a method for efficiently treating inflammatory diseases, in particular IBDs. More particularly, the invention falls within the context of the use of synthetic SOD mimics for combating oxidative stress, in particular treating inflammatory diseases, and aims to propose a solution to increase the bioavailability and efficacy of such active principles at their site of action, in particular in the bowel, compared to the solutions proposed by the prior art for administering such compounds.

The inventors have now discovered that these objectives can be achieved using microorganisms as vectors for the targeted delivery of SOD mimics in the gut. They have in particular discovered that a synthetic inorganic complex can be accumulated in microorganism cells, and that, when these cells are administered orally to a subject, and pass in the stomach, they protect the inorganic complex they contained from contact with the very acidic gastric juices, and, therefore, prevent the decomplexation which would otherwise have been induced by this contact. A high proportion of the inorganic complex administered to the subject is therefore advantageously intact when the cells reach their site of action in the intestine.

Thus, protected during the progression of the microorganism cells in the gastrointestinal tract, the inorganic complex is still intact, and retains its full superoxide dismutation catalytic capacity, when it reaches its site of action in the bowel.

A first object of the invention is therefore a chemically-modified microorganism, i.e., a microorganism modified by introduction therein of a chemical compound, said microorganism being selected from bacteria and yeasts, and containing a synthetic inorganic complex which is a superoxide dismutase mimic, for its use as a medicament or a dietary supplement.

Such a chemically-modified microorganism, wherein the SOD mimic is vectorized, is particularly easy to prepare, by simple passive penetration of the inorganic complex through the cell wall and its accumulation inside the cell. When it is administered orally to a subject, the cell wall advantageously protects the inorganic complex from the acid gastric conditions, and prevents its dissociation and degradation, so that it is advantageously delivered in the intestine with its full bioactivity, in particular its full ability to correct any hyperpermeability of the intestine induced by colitis. This bioactivity has in particular been confirmed by an assay carried out in a murine model of acute colitis.

In the context of an intended therapeutic application of the chemically-modified microorganism of the invention, involving the administration thereof to a subject, both the microorganism and the inorganic complex are advantageously chosen so as not to be harmful to said subject.

Therefore, the inorganic complex of the invention is preferably pharmaceutically and/or physiologically acceptable, meaning that it produces no adverse, allergic or other undesirable reaction when it is administered to a subject, in particular to a mammal and more particularly to a human.

The microorganism is advantageously chosen so as to be non-pathogen with respect to the subject to which it is intended to be administered, in particular non-pathogen to mammals and more particularly to humans. The microorganism is preferably commensal with respect to this subject.

Food-grade bacteria and yeasts are particularly preferred in the context of the invention. By food-grade microorganisms, it is herein meant the microorganisms:
listed in the QPS (“Qualified Presumption of Safety”) list of the European Food Safety Authority (EFSA), last update (“2022 QPS list”), available at the Knowledge Junction in Zenodo (https://doi.org/10.5281/zenodo.1146566)—EFSA has written guidelines for the safety assessment and characterization of microorganisms (including probiotics) to be used as feed additives or as production (i.e., delivery) organism (EFSA Panel on Additives and Products or Substances used in Animal Feed);
and/or listed in the “Generally Recognized as Safe (GRAS)” document from the US Food and Drug Administration, 6 Sep. 2019, Retrieved 30 Jan. 2021.

The microorganism used according to the invention may be deficient in SOD enzyme, or it may produce such an enzyme.

By superoxide dismutase (SOD) mimic/mimetic, it is herein, in a conventional way, referred to a synthetic low-molecular-weight (in particular, of molecular weight of less than 10 kDa, and possibly less than 5 kDa) compound able to catalyze the dismutation of superoxide in an aqueous medium, i.e., having a similar effect on protection from oxidative damage as SOD. More particularly, SOD mimics are able to perform catalytic redox cycle to both oxidize O₂⁻ into O₂ and to reduce O₂⁻ into H₂O₂. This implies that the redox potential of the redox couple that is involved lies between-0.18V/NHE and 0.89V/NHE (at pH 7).

It falls within the skills of the person skilled in the art to identify inorganic complexes that are SOD mimics.

To date, a huge diversity of such complexes, in particular manganese complexes, has been reported by the prior art. They involve ligands such as salen derivatives, cyclic polyamine, tri- or dipodal nitrogen-centered ligands, 1,2-ethanediamine-centered ligands, desferrioxamine derivatives, polyaminocarboxylato- or polycarboxylato ligands, peptides, porphyrins, phthalocyanines, texaphyrins, corroles or biliverdin and its derivatives, etc. Examples of SOD mimics, which can be used according to the invention, are in particular cited in the publication of Vincent et al., 2021, J. Inorg. Biochem. 219, 111431.

Otherwise, for determining whether a given inorganic complex is a SOD mimic, i.e., possesses an intrinsic SOD activity, the person skilled in the art can apply the so-called McCord and Fridovich indirect assay, which is commonly used to identify SOD mimics by a test of superoxide dismutation, and which is described in the publication of McCord and Fridovich, 1969, J. Biol. Chem. 244 (22), 6049-6065. Schematically, this assay comprises determining the catalytic rate (k_{cat}) for superoxide dismutation in a HEPES buffer (for example, at 50 mM, pH 7.4), the inorganic complex to be tested being in competition with a redox marker such as ferricytochrome c, 2,3-bis-(2-methoxy-4-nitro-5-sulfophenyl)-2H-tetrazolium-5-carboxanilide (XTT) or nitro blue tetrazolium (NBT), for example at 100 M, to react with superoxide that is continuously produced via an enzymatic xanthine (200 μM)/xanthine oxidase system. The reduction of the redox marker is monitored by UV-visible spectroscopy, for example at 550 nm for ferricytochrome c, for different amounts of the inorganic complex. Reduction rates in the absence and in the presence of the complex are both determined for each amount of the inorganic complex added. If s1 is the slope of the reduction curve before addition of the inorganic complex and s2 is the slope after addition thereof, for each inorganic complex the inhibition percentage is given by I (%)=(s1-s2)/s1×100. The value of the IC₅₀, which is the concentration of the inorganic complex required to induce a 50% inhibition of the reduction of the redox marker, is obtained for I=50%; (s1-s2)/s2 can also be plotted against the inorganic complex concentration, providing a linear correlation. The IC₅₀ is then obtained for (s1-s2)/s2=1. The k_{cat} of the inorganic complex is then obtained from the IC₅₀ and the known value of the k_{cat} of the redox marker (k_{redox}) (for example, for XTT, k_{XTT}=2.9×10⁴ M⁻¹·s⁻¹ at pH 7.4), according to the equation: k_{cat}=k_{redox}×[redox]/IC₅₀,
wherein [redox] is the concentration of the redox marker in the assay medium, for example 100 μM.

An inorganic complex can be qualified as a SOD mimic when the kinetics of dismutation of superoxide catalyzed by this complex, as measured in the above assay, i.e., the k_{cat}, are faster than the auto-dismutation of superoxide (self-dismutation, without any catalyst) at the same pH, as tabulated in the publication of Bielski et al., 1985, J. Chem. Phys. Ref Data, 14 (4), 1041-1100, for example faster than 6.10⁵ M⁻¹·s⁻¹ at pH 7.

The controls for the reliability of this assay, in particular in order to ascertain that the inorganic complex tested does not inhibit superoxide production by inhibiting the reaction of xanthine oxidase, can be performed as described in Durot et al., 2005, Eur. J. Inorg. Chem., 17, 3513-3523.

In particular embodiments of the invention, the inorganic complex is a mimic of human manganese superoxide dismutase.

More generally, the metal of the inorganic complex is preferably manganese (II) or manganese (III), which have the advantage, in particular compared to other metals such as iron, copper or nickel, of not increasing oxidative stress when released in the organism.

Any SOD mimic inorganic complex, in particular any manganese-based SOD mimic, described by the prior art, falls within the scope of the invention, in particular those having ligands such as salen derivatives, cyclic polyamine, tri- or dipodal nitrogen-centered ligands, 1,2-ethane-diamine-centered ligands, desferrioxamine derivatives, polyaminocarboxylato- or polycarboxylato ligands, peptides, porphyrins, phthalocyanines, texaphyrin, corroles or biliverdin and its derivatives, mentioned in the above-referenced publication from Vincent et al., 2021.

By way of examples, the microorganism of the invention can contain:
Mn(II) dichloro[(4aR,13aR, 17aR,21aR)-1,2,3,4,4a,5,6,12,13,13a,14, 15,16, 17,17a, 18,19,20,21,21a-eicosahydro-11,7-nitrilo-7Hdibenzo[b,h] [1,4,7,10] tetraazacycloheptadecine-kN5,kN13,kN18,kN21,kN22] (Imisopasem manganese), a particular manganese (II) pentaazamacrocyle (cyclic polyamine);
as Mn(III) porphyrins: Mn(III) meso-tetrakis(N-ethylpyridinium-2-yl) porphyrin (MnTE-2-PyP⁵⁺); Mn(III) meso-tetrakis(N-(2′-n-butoxyethyl)pyridinium-2-yl) porphyrin (MnTnBuOE-2-PyP⁵⁺); Mn(III) 5,10,15,20-tetrakis(4-benzoic acid porphyrin (MnTBAP);
Mn(III) 2,2′-[1,2-ethanediylbis(nitrilome-thylidyne)]bis[6-methoxy-phenol);
etc.

In particular embodiments of the invention, the inorganic complex is inspired by the MnSOD active site and is built on a ligand with a 1,2-N,N′-diaminoethane central scaffold functionalized with Lewis bases and in which the diamino structure is rigidified with a C-based cycle, in order to limit Mn-release. The inorganic complex can in particular have the general formula (I):

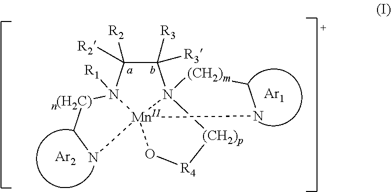

wherein:
n, m and p, identical or different, are integers between 1 and 3, each of n, m and p being preferably equal to 1,
R₁ represents a hydrogen atom or a linear, branched and/or cyclic alkyl, preferably having 1 to 12 carbon atoms, in particular 2 to 6 carbon atoms, optionally substituted by at least one, i.e., one or several, aromatic cycle(s) or polycycle(s), preferably having 6 to 14 carbon atoms, such as anthracene, or R₁ represents a peptide comprising from 1 to 20, in particular from 1 to 9, amino acid residues,
R₂, R₂′, R₃, R₃′ all represent a hydrogen atom,
or R₂′ and R₃′ represent a hydrogen atom and R₂ and R₃ form, together with the carbon atoms to which they are attached, an aliphatic cycle or heterocycle, in particular a 5-, 6-, 7- or 8-membered cycle or heterocycle, optionally substituted by a Lewis base, a phenol group, an imidazole ring, a pyridine ring, and/or by one or several identical or different alkyl group(s), each preferably having 1 to 12 carbon atoms, in particular 1 to 6 carbon atoms, each of said alkyl group(s) being optionally substituted by a Lewis base, a phenol group, an imidazole ring and/or a pyridine ring,
or R₂, R₂′, R₃, R₃′ form, together with the carbon atoms to which they are attached, an aromatic cycle or heterocycle, in particular a 5-, 6-, 7- or 8-membered cycle or heterocycle, optionally substituted by a Lewis base, a phenol group, an imidazole ring, a pyridine ring, and/or by one or several identical or different alkyl group(s), each preferably having 1 to 12 carbon atoms, in particular 1 to 6 carbon atoms, each of said alkyl group(s) being optionally substituted by a Lewis base, a phenol group, an imidazole ring and/or a pyridine ring,
Ar₁ and Ar₂, which may be identical or different, each represents a 5-membered or 6-membered aromatic heterocycle, preferably comprising one or two heteroatoms, in particular one or two nitrogen atoms, such as a pyrrole, an imidazole, a pyridine or a pyrimidine ring, said aromatic heterocycle being optionally substituted by one or several identical or different substituents, each of which is selected from a bromine atom, a chloride atom, a methyl group, a methoxy group and a nitro group,
R₄ represents a phenyl group, optionally substituted by one or several identical or different substituents, each of which is selected from a bromine atom, a chloride atom, a methyl group, a methoxy group and a nitro group,
or R₄ represents a group of formula —R₅—CO—, wherein the carbonyl group is attached to the oxygen atom and R₅ is attached to the —(CH₂)ₚ— group, and R₅ represents a linear, branched and/or cyclic alkyl, preferably having from 1 to 3 carbon atoms, optionally substituted by a Lewis base.

A Lewis base herein refers to a group which can donate a pair of electrons to an electron acceptor, such as manganese (II), so as to form a non-permanent coordinate covalent bond. Examples of Lewis bases that can be comprised in the inorganic compound of the invention are the groups of formulae —OH, —COOH, —SH, —NH₂, —NHRₐ, —NRₐ(R_{b}), wherein Rₐ and R_{b}, identical or different, each represent a C1-C3 alkyl or alkenyl group or a phenyl group.

All the stereoisomers, diastereoisomers and enantiomers of the inorganic complex of general formula (I) above, as well as of all the formulae below, fall in the scope of the invention, either taken individually or in any of the mixtures thereof. In particular, the nitrogen atoms respectively bound to the Cₐ and the C_{b} atoms can be in a cis configuration. They are preferably in a trans configuration. Any salt of the inorganic complex of formula (I), or of any of the formulae below), falls within the scope of the invention.

The inorganic complex of formula (I) can respond to one or several of the features below, in any of the combinations thereof.

R₁ can for example be a n-propyl group.

In particular embodiments of the invention, when at least R₂ and R₃ do not represent a hydrogen atom, then R₁ represents a hydrogen atom.

When Ar₁, respectively Ar₂, represents a substituted six-membered aromatic heterocycle, such as a pyridine or a pyrimidine ring, the substituent(s) are preferably in ortho position, and more preferably in para position, relative to the nitrogen atom situated in a position with respect to the carbon atom attached to —(CH₂)ₘ—, respectively —(CH₂)ₙ— (i.e., relative to the nitrogen atom coordinated with manganese (II)).

When Ar₁, respectively Ar₂, represents an imidazole ring, at least one of the substituents is bound to the nitrogen atom other than the one which is coordinated with manganese (II).

When R₄ represents a phenyl group, this group is preferably attached to —(CH₂)ₚ— at a carbon atom in α position with respect to the carbon atom bound to the oxygen atom.

The inorganic complex of the invention can have the general formula (II):

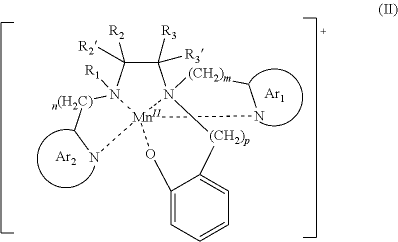

wherein m, n, p, R₁, R₂, R₂′, R₃, R₃′, Ar₁ and Ar₂ are as defined above.

It can in particular have the general formula (IIa):

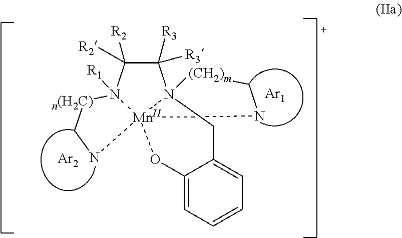

wherein m, n, R₁, R₂, R₂′, R₃, R₃′, Ar₁ and Ar₂ are as defined above.

Preferably, in formulae (II) and (IIa) above, m is equal to 1 and/or n is equal to 1. In particular embodiments of the invention, the inorganic complex has the general formula (III):

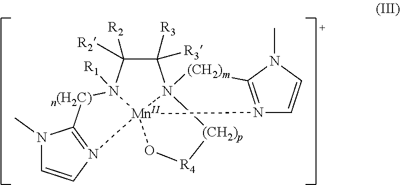

wherein m, n, p, R₁, R₂, R₂′, R₃, R₃′ and R₄ are as defined above.

It can in particular have the general formula (Illa):

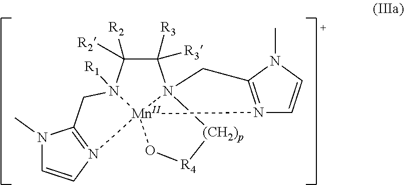

wherein p, R₁, R₂, R₂′, R₃, R₃′ and R₄ are as defined above.

Preferably, in formulae (III) and (Illa) above, p is equal to 1.

In particular embodiments of the invention, the inorganic complex has a coordination sphere consisting of two imidazole rings and one phenolate ion. The inorganic complex then preferably has the general formula (IV):

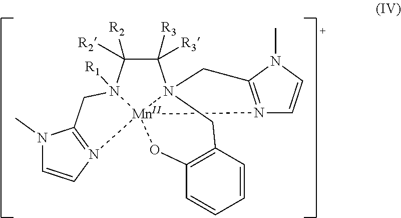

wherein R₁, R₂, R₂′, R₃ and R₃′ are as defined above.

A particular inorganic complex of formula (IV) is Mn(II)-(N-(hydroxybenzyl)-N,N-bis [2-(N-methyl-imidazolyl)methyl]-ethane-1,2-diamine)), herein called Mn1, of formula (V) above. In particular, Mn1 has the advantages of showing a clear anti-superoxide activity out of cellular context, as well as antioxidant and anti-inflammatory effects on intestinal epithelial cells and macrophages. As indicated above, Mn1 also ameliorates DNBS-induced colitis in a murine model, in particular according to the weight variation. The k_{cat} of Mn1, as obtained by the above-mentioned McCord and Fridovich assay, is 7.10⁶ M⁻¹·s⁻¹.

The inorganic complex of the invention can otherwise for example have one of the formulae (VI), (VII) and (VIII):

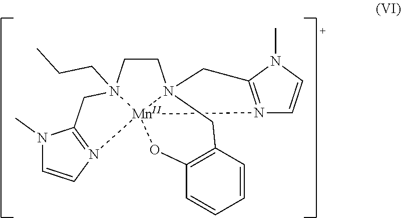

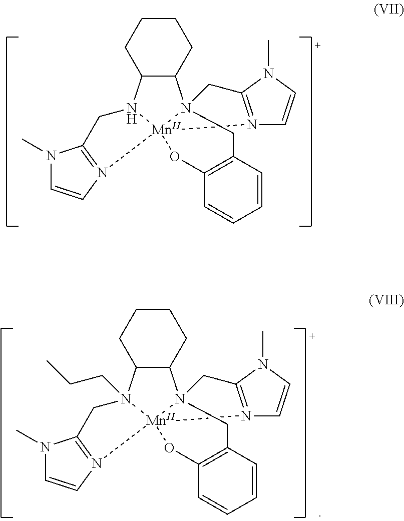

The inorganic complexes with a further rigidification of the 1,2-diaminoethane moiety, here exemplified with a 6-membered cyclohexyl moiety (complexes of formulae (VII) and (VIII) above), being advantageously more inert than Mn1 in the biological environment, are particularly preferred in the context of the invention.

The microorganism itself, chosen from bacteria and yeasts, is preferably selected among such microorganisms devoid of pro-inflammatory effect. In particular embodiments of the invention, the microorganism has an anti-inflammatory effect. Probiotics, with native beneficial properties, in particular anti-inflammatory properties, can be used in the context of the invention. Probiotics have been defined by the World Health Organization (WHO, 2001) as “live microorganisms that, when administered in adequate amounts, confer a health benefit on the host”. This definition as also been re-examined and validated more recently by a group of experts (Hill et al., Expert consensus document: The International Scientific Association for Probiotics and Prebiotics consensus statement on the scope and appropriate use of the term probiotic. Nat. Rev. Gastroenterol. Hepatol. 2014, 11, 506-514; Salminen et al. The International Scientific Association of Probiotics and Prebiotics (ISAPP) consensus statement on the definition and scope of postbiotics. Nat. Rev. Gastroenterol. Hepatol. 2021, 18, 649-667).

Moreover, microorganisms the cell-wall of which has a good permeability to inorganic complexes are favored in the context of the invention.

The microorganism of the invention can be a yeast, for example of the species Saccharomyces boulardii or Yarrowia lipolytica.

In alternative embodiments of the invention, the microorganism is chosen among bacteria, which have the advantage of being easy to manipulate and fast growing.

Bacteria naturally rich manganese, i.e., having a manganese content of approximately 1 mM or more, are particularly preferred in the context of the invention, as a high manganese content advantageously promotes the persistence, within the cells, of manganese in its ligand-coordinated form.

In particular embodiments of the invention, the microorganism is a lactic acid bacterium, in particular of the genera Lactobacillus or Lactococcus. Such bacteria combine in particular the advantages of being capable of surviving in a large range of pH, in particular in the acidic medium of the gastrointestinal tract, of a good penetration of inorganic complexes therein, and a high manganese content, which forces the ligand-metal coordination and maximizes the amount of assembled inorganic complex in the cell.

The bacteria can be selected from the genera Lactobacillus, Lactococcus, Bifidobacterium or Bacillus, the species Lactobacillus plantarum and Lactococcus lactis being particularly preferred.

The bacteria can also be bacteria of the species Escherichia coli which are non-pathogenic for mammals, in particular for humans.

In preferred embodiments of the invention, the microorganism is modified so as to improve the targeted and efficient release of the encapsulated inorganic complex in a suitable zone for obtaining therapeutic efficiency, more particularly at the inflammation site in the bowel. Preferably, this is achieved by weakening the microorganism's cell wall before administering it to the subject, so that when the chemically-modified microorganism of the invention is administered to the subject, after passing in the stomach and reaching the gut, during its transit therein, and as its wall has beforehand been fragilized by the previous weakening treatment, it advantageously lyses and liberates the SOD mimic inorganic complex in the bowel.

Therefore, in particular embodiments of the invention, the cell wall of the microorganism has been genetically, chemically and/or enzymatically weakened. As a consequence, this cell wall is more easily lysed than that of the original, non-weakened, microorganism. Generally speaking, cells the wall of which have been weakened grow more slowly and precipitate more quickly than their non-weakened counterparts. Microorganisms the cells of which have been weakened can for example be identified using a membrane permeability determination kit, such as the Baclight® kit, for measuring the ability of a reagent such as propidium iodide to penetrate the cells. Microorganisms the cell wall of which has been weakened have a greater accumulation of this reagent than original microorganisms with non-weakened cell wall.

Any method of weakening the cell wall of the microorganism, without killing it, known by the person skilled in the art, falls within the scope of the invention. The step(s) of weakening the cell wall of the microorganism can be carried out before, during or after, the step of contacting the inorganic complex with the microorganism so as to promote its penetration therein. Preferably, they are carried out before this contacting step. The penetration rate of the inorganic complex into the microorganism cells is then advantageously increased.

As an example, the microorganism can be cultivated in the presence of a cell-wall weakening agent, such as glycine or threonine, for a few hours, preferably for 6 to 18 hours, for example for approximately 12 hours.

In particular embodiments of the invention, the microorganism is auxotrophic for a given substance, preferably to an amino acid, in particular of the D series, and it has been cultivated in a culture medium devoid of said substance/amino acid for at least 1 hour, preferably at least 3 hours. Such a deprivation of the amino acid induces a weakening of the microorganism's cell wall. The amino acid can for example be alanine, phenylalanine or thymidine.

For example, the Lactobacillus plantarum strain MD007, deposited on Mar. 23, 2023 with the French National Collection of Microorganism Cultures (CNCM) at the Institut Pasteur (25 Rue du Docteur Roux, Paris, France) under the accession number CNCM I-5940 (identification reference LBH791-MD007), can be used in the context of the invention. This strain has been obtained by rendering the strain Lactobacillus plantarum ATCC BAA-793/NCIMB 8826 auxotrophic to alanine, as described in the publication of Palumbo et al., 2004, FEMS Microbiology Letters, 233:131-138. Preferably, this strain is used after an alanine deprivation of 1 to 5 hours.

Otherwise, the strain Lactococcus lactis MG1363 (which has an SOD enzyme) can be used in the context of the invention.

According to the invention, the chemically-modified microorganism is used as a medicament, for treating a disease, or as a dietary supplement.

The term “treating” as used herein means obtaining a desired pharmacological and physiological effect, which can be prophylactic or curative. The term “treating” as used herein therefore includes: preventing, or partially preventing, a disease, symptom or condition thereof, from occurring in a subject which has not yet been diagnosed as having the disease; and/or partially or completely curing a disease, symptom or condition thereof, or an adverse effect attributed to the disease.

The chemically-modified microorganism of the invention can be used for combating oxidative stress. By “combating” it is herein meant preventing or reducing oxidative stress.

The chemically-modified microorganism of the invention can be used for treating a wide range of pathophysiological processes wherein oxidative stress is involved, including diabetes, inflammatory and neurodegenerative diseases, reperfusion injury after ischemia, cancer, skin diseases associated with oxidative stress such as psoriasis, or any other disease related to oxidative stress.

The chemically-modified microorganism of the invention can in particular be used for treating inflammatory diseases, and more particularly inflammatory bowel diseases, such as Crohn's disease or ulcerative colitis.

The subject treated with the chemically-modified microorganism of the invention, who is suffering from the disease or is susceptible to contracting the disease, is preferably a mammal, in particular a human. It may also for example be a dog or a cat.

In preferred embodiments of the invention, the chemically-modified microorganism of the invention is administered to the subject orally.

It can otherwise be administered topically, especially in the context of the treatment of skin disease.

In the context of a medicament, the chemically-modified microorganism of the invention is preferably administrated to the subject in a therapeutically-effective amount, i.e., an amount that is suitable for delivering a therapeutically-effective amount of the SOD mimic inorganic complex to the subject.

By “therapeutically-effective amount” it is herein meant the amount of a compound which, when administered to a subject for treating a disease, is sufficient to perform such treatment of the disease. The therapeutically-effective amount of a compound depends on several factors, such as the disease and its severity, the age, weight, etc., of the subject to be treated, the particular compound used, the route and form of administration, etc. The amount of the chemically-modified microorganism of the invention to be administered to the subject will therefore be determined by the practitioner for each individual case.

It should in particular be low enough not to induce toxicity, such as manganism when the metal is manganese.

By way of example, a dosage equivalent to 0.10 to 0.20, for example 0.14, mg/kg/day of manganese can be applied.

In the context of a dietary supplement, the chemically-modified microorganism of the invention is preferably administrated to the subject in an amount that is suitable for delivering to the subject an amount of the SOD mimic inorganic complex which is suitable for obtaining a desired effect, such as a beneficial effect for health, comfort and/or well-being.

The chemically-modified microorganism of the invention can for example be administered to the subject one or twice a day. In particular, it can be administered to the subject twice a day, for example in mornings and evenings, during flare-ups of the disease, and once a day during remission phases.

The invention may also be expressed in the terms of a method of therapeutically treating a subject suffering from a disease, in particular from an inflammatory disease, more particularly an inflammatory bowel disease, such as Crohn's disease or ulcerative colitis, said method comprising administering to said subject in need thereof a therapeutically-effective amount of a chemically-modified microorganism of the invention. This method may have any of the features or combination of features described above in relation with the use as a medicament of a chemically-modified microorganism according to the invention. The chemically-modified microorganism may be administrated to the subject as a component of a medicament, or as a component of a food composition, a nutraceutical composition or a dietary supplement.

The invention also relates to the use of a chemically-modified microorganism according to the invention for the manufacture of a medicament, in particular a medicament for treating an inflammatory disease, more particularly an inflammatory bowel disease, such as Crohn's disease or ulcerative colitis. This use may respond to any of the features or combination of features described herein above in relation with the use as a medicament of a chemically-modified microorganism according to the invention.

The invention also relates to the use of a chemically-modified microorganism according to the invention for the manufacture of a food composition, a nutraceutical composition or a dietary supplement, in particular for combating oxidative stress. This use may respond to any of the features or combination of features described herein above in relation with the use as a medicament or a dietary supplement of a chemically-modified microorganism according to the invention.

The invention also relates to a pharmaceutical composition containing a chemically-modified microorganism as defined above, in a pharmaceutically suitable vehicle. In this pharmaceutical composition, the active principle is the SOD mimic inorganic complex encapsulated in the microorganism cells.

A “pharmaceutically suitable vehicle” herein means a vehicle that is useful in preparing a pharmaceutical composition or formulation and that is generally safe, non-toxic, and neither biologically nor otherwise undesirable for the subject to be treated, in particular mammals and more particularly humans.

The vehicle of the composition of the invention may be solid, semi-solid or liquid. It may be a diluent, an adjuvant or any other vehicle conventional per se for the constitution of pharmaceutical compositions.

The chemically-modified microorganism can be contained therein in any form, in particular in a lyophilized form.

The pharmaceutical composition of the invention may be formulated in any galenical form, in particular in a form that is suitable for administration in mammals, and in particular in humans. It is preferably formulated in a form which is suitable for administration by oral route, such as a form of powder, capsules, oral solution or suspension, or in a form which is suitable for administration by topical route, especially on the skin and/or the mucous of the subject, such as a cream, etc.

The pharmaceutical composition of the invention may comprise one or more excipients/additives conventional by themselves, for example preservatives, sweetening agents, flavoring agents, suspending agents, dispersing agents, lubricating agents, stabilizing agents, buffering agents, or any of their mixtures. It may also contain one or more other active agents, which may or may not act in synergy with the inorganic complex of the invention, for example other anti-inflammatory agent(s) and/or a pain-relieving agent(s).

The invention also relates to the therapeutic use of a pharmaceutical composition as defined above, in particular for treating an inflammatory disease, more particularly an inflammatory bowel disease, such as Crohn's disease or ulcerative colitis in a subject, in particular a mammal and more particularly a human.

The invention also relates to a nutraceutical composition, food composition or dietary supplement, containing a chemically-modified microorganism as defined above, in a physiologically suitable vehicle. In this composition/supplement, the active principle is the SOD mimic inorganic complex encapsulated in the microorganism cells.

A “physiologically acceptable vehicle” herein means a vehicle that is physiologically tolerable and does not produce an allergic or similar unwanted reaction when administered to a subject, in particular a mammal and more particularly a human.

The vehicle of the composition/supplement may be solid, semi-solid or liquid. It may be a diluent, an adjuvant or any other vehicle conventional per se for the constitution of nutraceutical compositions, food compositions or dietary supplements.

The chemically-modified microorganism can be contained therein in any form, in particular in a lyophilized form.

The nutraceutical composition, food composition or dietary supplement of the invention is preferably in a form which is suitable for oral administration.

It may comprise one or more probiotics, prebiotics, vitamins, polyphenols, minerals, materials suitable for oral administration (of liquid or gel type, such as a solvent, a diluent, a non-toxic solubilizing agent which does not interact with the components of the composition in a deleterious manner), and/or any other ingredient or excipient such as proteins, amino acids, carbohydrates, lipids, oligosaccharides, other micronutrients, metal salts or cations, etc.

The invention also relates to the use of a nutraceutical composition, food composition or dietary supplement as defined above for combating oxidative stress and/or for treating an inflammation in a subject, in particular a mammal and more particularly a human.

A method of producing a chemically-modified microorganism according to the invention comprises contacting cells of the microorganism with a composition containing said superoxide dismutase mimic inorganic complex, preferably for at least 1 hour, to a few hours, in order to allow the inorganic complex to penetrate, by a passive mechanism, into the microorganism cells and accumulate therein. This contacting step is preferably carried out:
for a period of between 1 and 12 hours preferably between 1 and 5 hours, for example of about 2 hours;
and/or at 37° C.;
and/or under shaking;
and/or in a liquid composition, preferably with a pH of between 7 and 8, such as a solution of 4-[2-(hydroxyethyl)-1-piperazin-1-yl]ethanesulfonic acid (HEPES) 0.1 M;
and/or when the optical density of the cell suspension at 600 nm is between 0.6 and 0.8.

The concentration of the inorganic complex in the liquid composition is then preferably comprised between 0.1 to 5 mM, for example substantially equal to 0.4 mM. Such a concentration range is advantageously high enough to obtain the penetration of a high amount of the inorganic complex in the microorganism cells, and low enough to avoid accumulation therein of a high amount of other forms of the metal, for example, when the metal is manganese, of manganese in the form of MnCl₂, while ensuring a lack of toxicity for the cells.

As explained above, the method can comprise, before, during or after the step of contacting the cells of the microorganism with a composition containing said superoxide dismutase mimic inorganic complex, preferably before this step, a step of weakening the cell wall of the microorganism.

Such a step can be carried out by any method known by the person skilled in the art, for example by cultivating the microorganism in a culture medium supplemented with a cell-wall weakening agent such as glycine, for example at a concentration of between 10 and 20 g/L, for example for a period of between 1 to 12 hours. The culture medium may be any medium suitable for cultivating the microorganism. It includes in particular all the nutrients and other elements necessary for the survival, and optionally growth, of the microorganism.

In the particular embodiments of the invention wherein the microorganism is auxotrophic for a given amino acid, such as alanine, the method comprises a cell-wall weakening step of cultivating said microorganism in a culture medium devoid of said amino acid. The culture medium includes at least all the nutrients and other elements necessary for the survival, and optionally growth, of the microorganism, such as a De Man, Rogosa and Sharpe (MRS) medium, and is devoid of said amino acid. Such a deprivation step can be carried out for a period of between 1 and 5 hours, for example of about 3 hours. It is preferably started when the optical density at 600 nm of the culture has reached 0.6, and before the step of contacting cells of the microorganism with a composition containing the superoxide dismutase mimic inorganic complex.

In all cases, the cultivating step is preferably carried out at 37° C. An example of a method for preparing a chemically-modified auxotrophic microorganism according to the invention comprises successive steps of:
cultivating the microorganism, in particular the bacteria, preferably at 37° C., in a culture medium, such as MRS, supplemented with the substance for which the microorganism is auxotrophic, until an optical density at 600 nm of between 0.6 and 0.8 is obtained;
separating the cells from the culture medium, for example by centrifugation, and optionally washing the cells, for example with MRS medium, preferably three times;
cultivating the cells in MRS medium devoid of said substance, for 1 to 5 hours, for example for 3 hours (“substance deprivation”);
separating the cells from the culture medium, for example by centrifugation, and optionally washing the cells, for example with a solution of HEPES 0.1M, preferably three times;
resuspending the cells in a liquid solution, for example a solution of HEPES 0.1M, containing the SOD mimic inorganic complex, preferably at a concentration of between 0.1 to 5 mM, for example of 0.4 mM;
incubating the suspension at 37° C. for 1 to 5 hours, for example for 2 to 3 hours, preferably with shaking;
separating the cells from the liquid medium, for example by centrifugation, and optionally washing the cells, for example with a solution of HEPES 0.1M;
and finally recovering the cells, for example in a 16% glycerol-HEPES 0.1M solution.

The chemically-modified cells thus obtained, containing a high amount of the SOD mimic inorganic complex, may then be administered to a subject in need thereof, for preventively or curatively treating a disease related to oxidative stress, in particular an inflammatory bowel disease. This administration should then preferably be performed quickly after the obtention of the chemically-modified cells, more particularly a few hours after their obtention.

Otherwise, the cells can be lyophilized, and used as such; or they can be frozen, for example at −80° C., in a buffer such as a HEPES buffer supplemented with glycerol, for example at 16% v/v, and thawed extemporaneously before being administered to the subject to be treated.

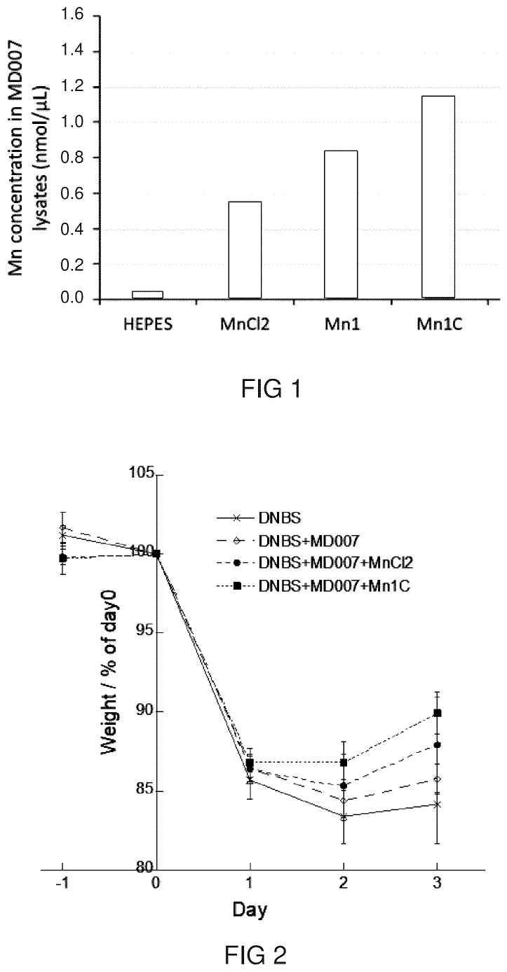

The features and advantages of the invention will emerge more clearly in the light of the following examples of implementation, provided for illustrative purposes only and in no way limitative of the invention, with the support of FIGS. 1 to 14, in which:

FIG. 1 shows a bar graph representing the Mn concentration measured by ICP-MS in cell lysates of bacteria MD007 empty (“HEPES”) or loaded with SOD mimics (Mn1, Mn1C) or with MnCl₂;

FIG. 2 shows a graph representing the weight monitored over time of mice subjected to a DNBS injection (at Day 0) and treated everyday orally with MD007 bacteria empty or loaded with a SOD mimic (Mn1C) or with MnCl₂ (from Day-1 to Day 2); the weights are expressed as a percentage of the mice initial weight before the start of the assay;

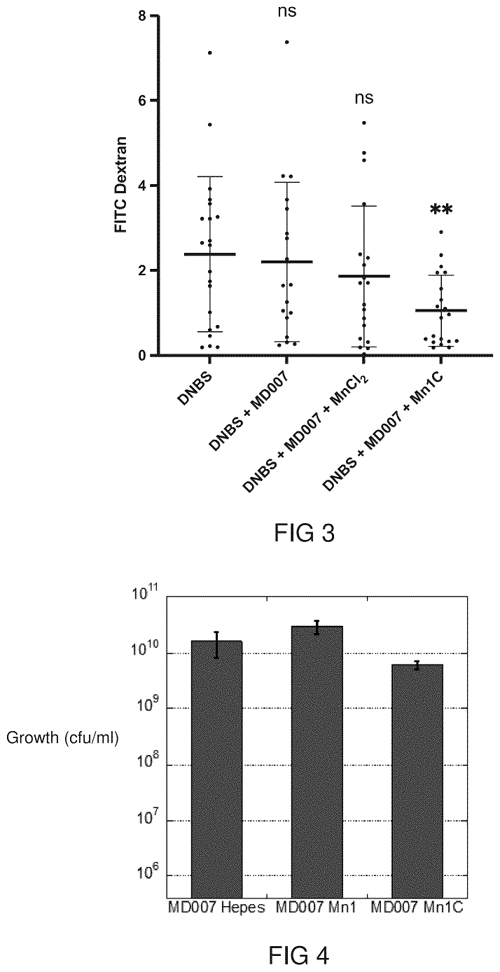

FIG. 3 shows a graph representing the quantification of FITC-dextran as a marker of the intestinal barrier permeability in mice having been subjected to DNBS injection and treated orally with MD007 bacteria empty or loaded with a SOD mimic (Mn1C) or with MnCl₂. Data represent the mean±SD for three independent assays (around 24 mice). The p-values were calculated using the non-parametric Mann-Whitney test (one-tailed test) as the gaussian distribution of the data was unvalidated by the Anderson-Darling normality test. The mean ranks of each column were compared to that of the DNBS control; each comparison stands alone. **: p<0.01 versus DNBS control, and ns means nonsignificant;

FIG. 4 shows a graph bar representing the enumeration of viable cells in frozen-thawed aliquots of MD007 bacteria empty (“HEPES”) or loaded with SOD mimics (Mn1, Mn1C);

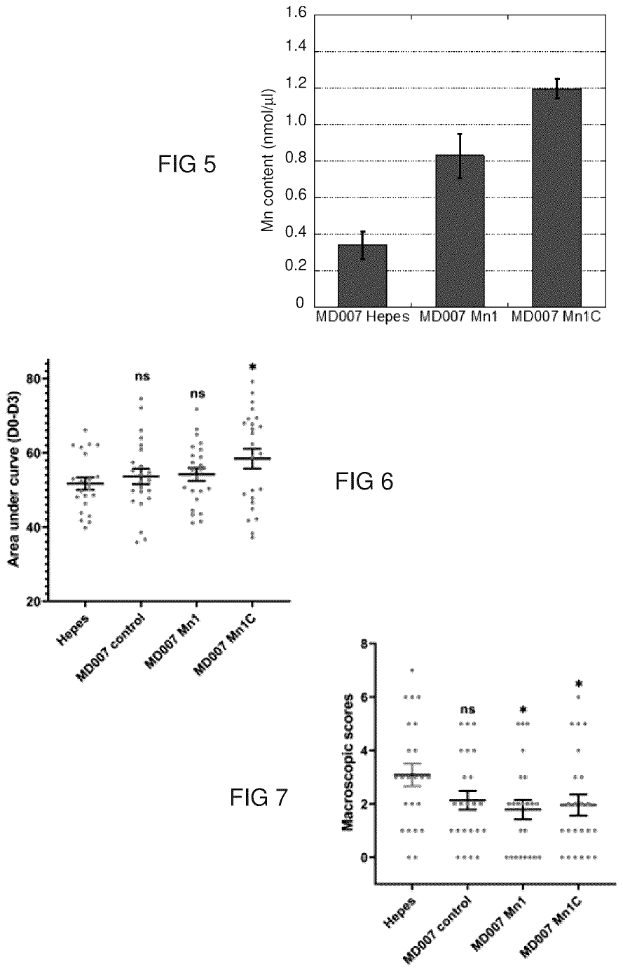

FIG. 5 shows a bar graph representing the Mn content measured by ICP-MS in cell lysates of bacteria MD007 empty (“HEPES”) or loaded with SOD mimics (Mn1, Mn1C) (means of 3 experiments);

FIG. 6 shows a graph representing the area under the curve of weight monitoring between day 0 and day 3 (D0-D3) of mice subjected to a DNBS injection (at Day 0) and treated every day orally with Hepes (“Hepes”), MD007 bacteria empty (“MD007 control”) or loaded with SOD mimics Mn1 or Mn1C (data collected from 3 independent assays, with 8 mice/assay/condition);

FIG. 7 shows a graph representing the macroscopic scores, determined on the euthanasia day, of mice subjected to DNBS injection and treated every day for 4 days orally with Hepes (“Hepes”), MD007 bacteria empty (“MD007 control”) or loaded with SOD mimics Mn1 or Mn1C (data collected from 3 independent assays, with 8 mice/assay/condition);

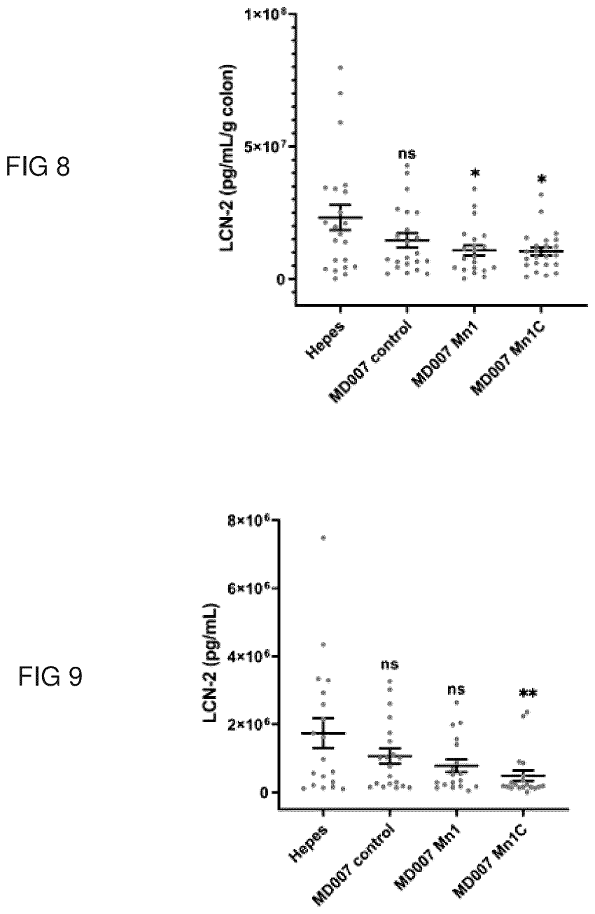

FIG. 8 shows a graph representing the concentration of lipocalin 2 (LCN-2) measured in the colon of mice subjected to DNBS injection and treated every day for 4 days orally with Hepes (“Hepes”), MD007 bacteria empty (“MD007 control”) or loaded with SOD mimics Mn1 or Mn1C (data collected from 3 independent assays, with 8 mice/assay/condition);

FIG. 9 shows a graph representing the concentration of lipocalin 2 (LCN-2) measured in the serum of mice subjected to DNBS injection and treated every day for 4 days orally with Hepes (“Hepes”), MD007 bacteria empty (“MD007 control”) or loaded with SOD mimics Mn1 or Mn1C (data collected from 3 independent assays, with 8 mice/assay/condition);

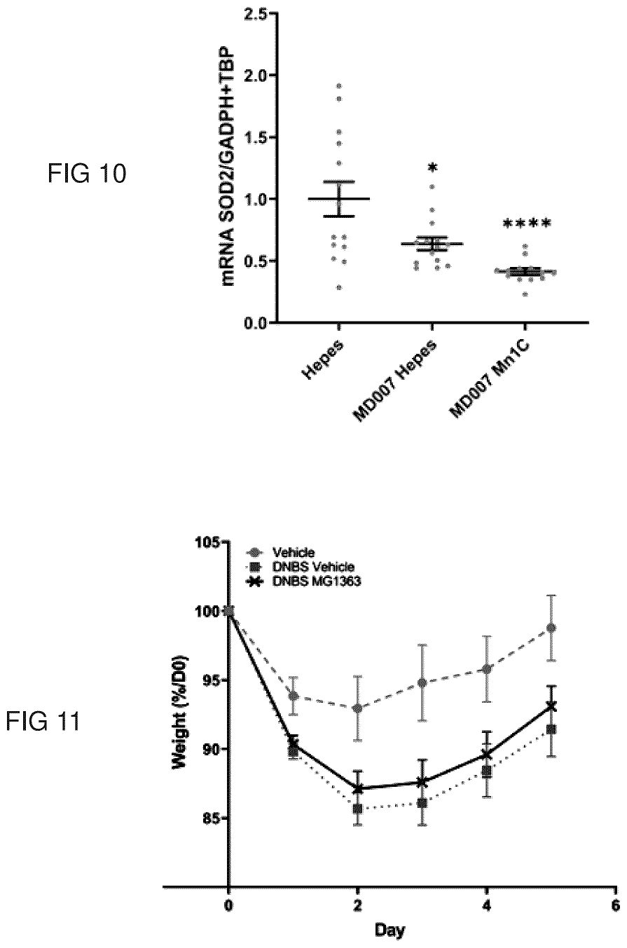

FIG. 10 shows the levels of SOD2 mRNA, normalized with those of GADPH and TBP, measured in colon samples of mice subjected to DNBS injection and treated every day for 4 days orally with Hepes (“Hepes”), MD007 bacteria empty (“MD007 Hepes”) or loaded with the SOD mimic Mn1C (average on 16 mice per condition);

FIG. 11 shows a graph representing the weight monitored over time of mice subjected to a DNBS injection (at Day 0) or not (“Vehicle”) and treated every day orally with Hepes (“DNBS Vehicle”) or with empty MG1363 bacteria (“DNBS MG1363”)—the weights are expressed as a percentage of the mice initial weight before the start of the assay;

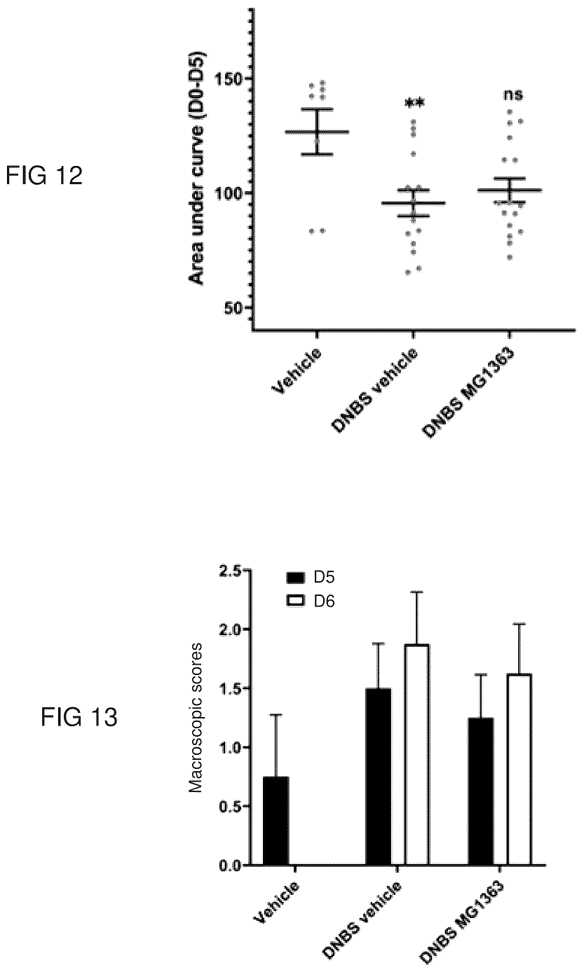

FIG. 12 shows the area under the curve of the curves of FIG. 11 (between day 0 and day 5: D0-D5);

FIG. 13 shows a graph representing the macroscopic scores, determined on the euthanasia day (day 5: “D5” or day 6: “D6”), of mice subjected to DNBS injection and treated every day for 5 days orally with Hepes (“DNBS Vehicle”) or with empty MG1363 bacteria (“DNBS MG1363”);

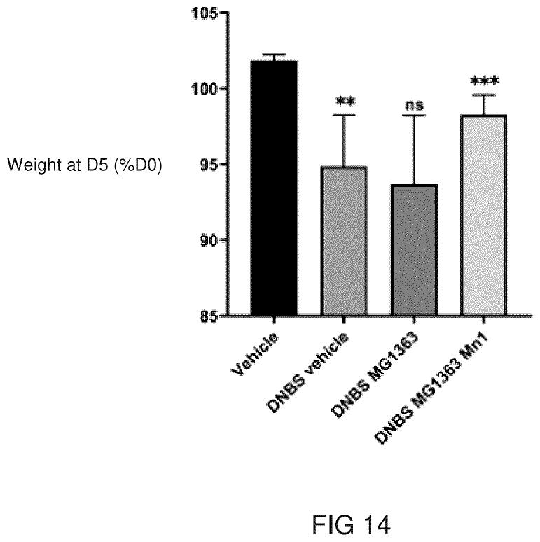

and FIG. 14 shows a graph bar representing the weight at day 5 (D5) of mice subjected to a DNBS injection (at Day 0) or not (“Vehicle”) and treated every day orally with Hepes (“DNBS Vehicle”), with empty MG1363 bacteria (“DNBS MG1363”) or with MG1363 bacteria loaded with Mn1—the weights are expressed as a percentage of the mice initial weight before the start of the assay (DO).

### EXAMPLE 1

### Synthesis of SOD Mimics Inorganic Complexes

The ligands used to prepare the inorganic complexes were EnPI2C and EnPI2CP, of respective formulae (Ixa) and (Ixb):

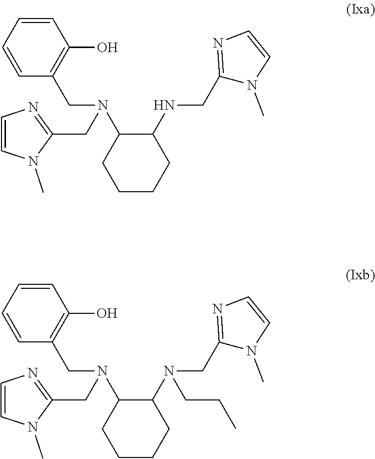 [SMILES: Cn1ccnc1CNC1CCCCC1N(Cc1ccccc1O)Cc1nccn1C ; CCCN(Cc1nccn1C)C1CCCCC1N(Cc1ccccc1O)Cc1nccn1C]

The SOD mimics Mn(II) complexes were prepared by mixing these respective ligands and MnCl₂ in a molar ratio 1:1.3 in HEPES 0.1 M (pH 7.5) and let 2 h at ambient temperature to afford a complete complexation of the ligands with manganese (II).

The following SOD mimics metal complexes were obtained: Mn1C (of formula (VII) above) from EnPI2C, Mn1CP (of formula (VIII) above) from EnPI2CP.

### EXAMPLE 2

Bacteria Loading with the SOD Mimics

The L. plantarum strain MD007, deposited on Mar. 23, 2023 with the French National Collection of Microorganism Cultures (CNCM) at the Institut Pasteur (25 Rue du Docteur Roux, Paris, France) under the accession number CNCM I-5940 (identification reference LBH791-MD007), was used for the experiments. This strain was obtained by rendering the strain Lactobacillus plantarum ATCC BAA-793/NCIMB 8826 auxotrophic to alanine, as described in the publication of Palumbo et al., 2004, FEMS Microbiology Letters, 233:131-138.

Overnight culture of MD007 cells in MRS medium supplemented with alanine (MRS-alanine) was diluted to OD_{600 nm}=0.2 in MRS-alanine and the cells were left to grow until reaching OD_{600 nm}=0.6. The culture was then centrifugated and the pellet was washed twice and resuspended in MRS without alanine.

The bacteria were deprived of alanine for 3 hours to fragilize them for the purpose of accelerating their lysis in mice intestine.

After alanine deprivation, the culture was again centrifugated and the pellet was washed twice and resuspended in HEPES 0.1 M. At that time, the culture was split into 4 parts and the SOD mimics Mn1, Mn1C and Mn1CP, and MnCl₂ were added respectively therein to a final concentration of 0.4 mM. The bacterial suspensions were let to incubate for 2 hours at 37° C. with agitation.

Lastly, after cultures centrifugation and washing with HEPES 0.1 M, the bacteria were resuspended in HEPES 0.1 M containing 16% glycerol (HEPES-gly) and stored at −80° C. in 2 mL aliquots to avoid freeze-thaw cycles.

### EXAMPLE 3

### Mn Quantification by ICP-MS Analysis

For each inorganic complex, and for the control MnCl₂, one of the aliquots of the bacterial suspension (at OD_{600 nm}=1) was used for Mn quantification by ICP-MS (Inductively Coupled Plasma Mass Spectrometry. A negative control with unloaded bacteria (HEPES) was also analyzed.

⁵⁵Mn was selected as isotope to avoid isobaric interferences.

ICP-MS experiments were performed on a 7700 Series ICP-MS system with an ASX-500 Series autosampler from Agilent, with the following parameters: RF power: 1550 W; sampling depth: 10 mm; helium flow rate: 5 mL/min, measurement, replicated five times with 100 sweeps per replicate, and an integration time/mass of 1 s.

The bacterial suspensions were diluted in 2% HNO₃, to lyse the bacteria and free Mn from all coordination sites. To achieve complete bacterial lysis, the 2% HNO₃ solutions were left for 1 h at ambient temperature and then filtered. A calibration curve was established using a commercial multi-element standard and the total metal amount was normalized by the bacterial suspension OD_{600 nm}.

The results obtained are shown in FIG. 1, for HEPES, MnCl₂, Mn1 and Mn1C. They show a high manganese content in the bacteria loaded with the SOD mimics according to the invention.

### EXAMPLE 4

### Induction of DNBS Colitis in Mice and Bacteria Administration

The in vivo assays were conducted on pathogen-free male C57BL/6 mice in the animal facilities of the National Institute of Agricultural and Environmental Research (INRAE, Jouy-en-josas).

The timeline for colitis induction and mice gavage with the loaded bacteria was as follows. The assay lasted 5 days. The loaded bacteria were intragastrically administrated daily to the mice for the first 4 days. On the second day, mice were anesthetized intraperitoneally and colitis was induced by an intra-rectal injection of dinitrobenzene sulfonic acid (DNBS) (2.75 mg per mouse in 20 μl of PBS-ethanol (70/30 v/v)) through a plastic tube inserted 4 cm into the colon. The fifth and last day the mice were euthanized by cervical dislocation. Mice were weighted every day.

The studied groups are as follows: control colitis group (DNBS+HEPES-gly), empty bacteria control group (DNBS+MD007), and 2 groups of loaded bacteria: “DNBS+MD007_MnCl₂” and “DNBS+MD007_Mn1C”. Each group was composed of 8 mice. The assay was repeated three times independently meaning that, in total, 24 mice for each condition were used.

The amount of loaded bacteria given to the mice was determined in such a way that all mice receive the same amount of total manganese, fixed to 150 nmol per dose. This corresponds to a number of bacteria varying between 5×10⁸ to 2×10⁹ CFU. The amount of MD007 given to the control “DNBS+MB007” group was chosen equal to the maximal amount of viable loaded bacteria given to mice from the loaded bacteria groups. Lastly, the control colitis group received 200 μL of HEPES-gly.

### EXAMPLE 5

### Evaluation of Colitis Severity

### 5.1/ Mice Weight

The evolution of the mice weight throughout the assay is shown in FIG. 2.

As can be seen on this figure, the mice feeding with MD007-internalized SOD mimic Mn1C has allowed to limit the weight loss of mice and fasten weight recovery. Indeed, the weight reduction (maximal at day 2) reached only 13% and mice recovered about 90% of their initial weight at day 3.

Moreover, the mice treated with empty MD007 lost very slightly less weight than untreated mice suggesting a small probiotic activity of the strain.

### 5.2/ Post-Euthanasia Mice Dissection

The abdominal cavity of euthanized mice was opened and the colon was removed, opened longitudinally and washed from feces.

The colon was then longitudinally separated in pieces for the subsequent experiments.

### 5.3/ Intestinal Permeability Assay

Three hours before mice euthanasia, the mice received by gavage a fluorescent marker: fluorescein isothiocyanate (FITC)-dextran (12 mg per mice in 200 μl of PBS). Just before mice euthanasia, blood was collected in the presence of heparin via the puncture of the sub-mandibular vein. The collected total blood was then centrifuged to recover plasma. The measurement of fluorescence intensity allowed to determine FITC-dextran concentration in the plasma samples. To this end, a fluorescence reading was performed with a spectrofluorometer (excitation wavelength=488 nm, emission reading=520 nm) in a microplate, using 80 μl of plasma sample.

The results are shown in FIG. 3.

The DNBS injection is known to provoke an impairment of the intestinal tight junctions resulting in enhanced barrier permeability, which favors passage of FITC-Dextran from the gastrointestinal tract to the blood system. Significantly lower levels of FITC-dextran were quantified in the plasma samples of mice treated with MD007+Mn1C compared to untreated mice. This demonstrates the efficacy of MD007-vectorized Mn1C to attenuate significatively the DNBS-induced damages on intestinal permeability.

### EXAMPLE 6

Additional Experiments with the L. plantarum Strain MD007

### 6.1/ Loaded Bacteria Viability and Mn Quantification in Bacteria

Three independent experiments are carried out as described below. The results shown are the mean of these three experiments.

The Lactobacillus plantarum MD007 strain is grown in MRS containing D-alanine (200 μg/mL) at 37° C.

An overnight culture is used to inoculate fresh medium in order to get an OD_{600 nm} of 0.2, and cells are grown to an OD_{600 nm} of 0.7 and are washed three times D-alanine free MRS medium. The washed bacteria are incubated in medium without D-alanine for 3 h at 37° C. before SOD mimic loading. After alanine starvation, the bacteria are washed three times with Hepes (0.1 M, pH 7.4) to reach a concentration about 10⁹ bacteria per 200 μL.

After removal of the culture medium supernatant, bacteria are incubated with Hepes (0.1 M, pH 7.4) only, or with the tested compounds (Mn1 or Mn1C) at the desired concentration in the same buffer for two or twenty-four hours, at 37° C. Mn1C, Mn1 solutions are prepared extemporaneously to prevent degradation and manganese oxidation reactions. After cultures centrifugation and washing with Hepes (0.1 M, pH 7.4), the bacteria are resuspended in Hepes 0.1 M containing 16% glycerol to reach a concentration about 10⁹ bacteria per 200 μL. This volume corresponds to the maximal and usual volume administrated by gavage to mice every day. The bacteria are stored at −80° C. in 2 mL aliquots to avoid freeze-thaw cycles. Samples dedicated to mice gavage are frozen at −80° C. and warmed up to room temperature before being administrated to mice.

To assess bacteria viability, the overnight culture and the frozen vials are controlled by CFU (colony forming unit) test by diluting the preparation in PBS by serial 1/10 dilution and plating a known volume of the dilutions on the corresponding medium. The plates are incubated at 37° C. for 48 h. Colonies are then counted by visual observation. The results obtained are shown in FIG. 4. It can be seen that bacteria loading with Mn1 or Mn1C does not affect cell viability.

Quantification of manganese in bacterium lysates is carried out by ICP-MS. For ICP-MS analysis, bacteria pellets recovered by centrifugation are acidified in 2% HNO₃, to lyse cells and free Mn from all coordination sites. Samples are carefully filtrated on 0.2 μm sized filters to get rid of bacteria debris before analysis. A calibration curve is established using a commercial Mn standard. A range of concentration going from zero to 100 ppb is generally used for calibration. The results are shown in FIG. 5. They show a high manganese content in the bacteria loaded with the SOD mimics according to the invention.

### 6.2/ Mice Treatment

Male C57BL/6 mice (6 weeks old) were maintained under specific pathogen-free conditions (SPF) conditions. Mice were housed under standard conditions for a minimum of 1 week before experimentation for their acclimatation. All animal studies were conducted in accredited research facilities and approved by local ethics committees in addition to the French government (authorization no: 16744-201807061805486_v2).

The gavage of these mice with empty or loaded bacteria were started the day before inflammation induction by DNBS (D-1) to ensure that bacteria are already present in the colon. At DO, mice were anesthetized with an intra-peritoneal injection of a mix of ketamine (75 mg/kg, Imalgene, Boehringer Ingelheim Animal Health) and xylazine (9 mg/kg, Rompun, KVP). DNBS (3 mg/mouse in 30% ethanol/PBS) was administrated by an intrarectal injection using a catheter at about 3.5 cm into the colon. Bacteria (about 10⁹ bacteria/200 μL), loaded bacteria (CMB) or Hepes were daily intragastrically administrated to mice for 4 or 5 days. The amount of CMB given to the mice was determined in such a way that all mice receive the same amount of total manganese, fixed at 150 nmol per dose, which corresponds to a dose ranging from 1×10⁹ and 2×10⁹ bacteria. The amount of bacteria given to the DNBS MD007 control group was determined to correspond to the maximal number of bacteria given to the groups treated with CMB. Mice were supervised all along the experiment with special attention. The last day (D3 or D4), mice were euthanized by cervical dislocation. The studied groups are named as follows: colitis group without bacteria that received Hepes buffer instead (“Hepes”), a colitis group that received empty bacteria (“MD007 control”) and 2 colitis groups treated with CMB: “MD007 Mn1” and “MD007 Mn1C”. Each group was composed of 8 mice. The assay was repeated three or four times independently meaning that, in total, 24 or 32 mice for each condition were used.

The weight of all mice was monitored over time. The area under the curve of weight monitoring between day 0 and day 3 (D0-D3) is shown in FIG. 6. As can be seen on this figure, the mice feeding with MD007-internalized SOD mimics Mn1 and Mn1C has allowed to limit the weight loss of mice, and even increased the weight of the mice.

### 6.3/ Evaluation of Colitis Severity in the Treated Mice

After the treatment with the loaded bacteria, the abdominal cavity of each euthanized mice was opened and the colon was removed, opened longitudinally and washed from feces. The macroscopic score was then immediately assessed. This score evaluates the state of the colon by taking into account: thickening of colon wall, presence of ulcers, hyperemia, adhesion to other intra-abdominal tissues and consistency of colon content (indicator of transit anomalies). The results obtained are shown in FIG. 7. The treatment with bacteria loaded with Mn1 or Mn1C significatively improves the macroscopic score.

The colons were then longitudinally separated in two pieces. The right parts were laterally separated in 4 parts intended for subsequent analysis.

Lipocalin 2 (LCN-2) concentration was measured in the colon and in the plasma of the mice. For determination of plasma LCN-2 concentration, centrifuged blood samples (2000 g for 10 min at 20° C.) were used. A commercially available sandwich enzyme-linked immunosorbent assay (Mouse Lipocalin-2/NGAL DuoSet ELISA; R&D Systems Europe) was used according to the manufacturer's instructions. For determination of colon LCN-2 concentrations, colon supernatants were used with the same kit as for plasma. To this end, colon pieces were weighed and mechanically dissociated in 0.5% HTAB (hexadecyl trimethyl ammonium bromide) buffer using a Precellys® (Bertin) device (3 cycles of 20 s at 10 000 rpm) in presence of a mix of 1.4 and 2.8 mm ceramic beads. Samples were then briefly centrifuged and supernatants were collected. The results obtained are shown, respectively, on FIG. 8 for the LON-2 concentration in the colon and on FIG. 9 for the LON-2 concentration in serum. They show that treatment of the mice with bacteria loaded with Mn1 or Mn1C result in a significative decrease of lipocalin 2 concentration.

The transcription of the SOD2 gene was quantified on colon parts as follows. Total RNA was extracted from colon homogenates by using a Qiagen RNeasy kit according to the manufacturer's instructions. β-mercaptoethanol was used as reducing agent for the irreversible denaturation of RNases enzymes. Reverse transcription was then performed to produce complementary DNA with the oligo (dT) 12-18 primer and SuperScript® II reverse transcriptase enzyme (Invitrogen®). SOD2 gene expression was quantified by quantitative PCR on a StepOne® Real-time PCR system using Takyon® rox Sybr mastermix DTTP blue (Eurogentec). The following primer pair was:

(forward)
(SEQ ID No: 1)
5′-ATTAACGCGCAGATCATGCA-3′
and
(reverse)
(SEQ ID NO: 2)
5′-TGTCCCCCACCATTGAACTT-3′

and the annealing temperature was set to 60° C.

The SOD2 levels were normalized with those of housekeeping GAPDH and TATA-box binding protein (TBP) genes, which are known to be not impacted in inflammatory conditions, also quantified by RT-PCR. The results obtained, for bacteria loaded with Mn1C, are shown in FIG. 10. It is observed that the level of SOD2 mRNA is much lower for mice treated with bacteria loaded with Mn1C.

### EXAMPLE 7

Lactococcus lactis MG1363 Strain

Lactococcus lactis MG1363 strain, described in Sanders et al., 1995, J. Bacteriol 177:5254-5260, is used in this experiment. This strain has a SOD enzyme.

### 7.1/ Bacteria Loading

The strain is grown in M17 glucose at 30° C.

An overnight culture is used to inoculate fresh medium in order to get OD_{600 nm} Of 0.2, and cells are grown to an OD_{600 nm} of 0.7 The bacteria are washed three times with Hepes (0.1 M, pH 7.4) to reach a concentration about 10⁹ bacteria per 200 μL.

The bacteria are loaded with Mn1 using the protocol described in Example 6.

### 7.2/ Evaluation of Bacterial Neutrality Towards DNBS Induced Colitis

Male C57BL/6 mice (6 weeks old) as described in Example 6 were used in this experiment.

The first day of the assay (DO), mice were anesthetized with Ketamine and Xylazine 0.06% by intraperitoneal injection (IP). A DNBS solution (3 mg/mouse) in 30/70 ethanol/PBS v:v was administrated by an intrarectal injection (IR) using a catheter at about 3.5 cm into the colon. Bacteria suspension (10⁹ bacteria/200 μL) or Hepes (100 μL) were daily intragastrically administrated to mice for 5 days. Mice were supervised all along the experiment with special attention. The last day (D5) or the following day (D6), mice were sacrificed by cervical dislocation.

The weight of all mice was monitored over time. The evolution of the mice weight throughout the assay, from day 0 to day 5, is shown in FIG. 11. The areas under the curves are shown in FIG. 12. As can be seen on these figures, the administration of MG1363 empty to the mice with DNBS induced colitis does not significatively impact the mice weight.

The macroscopic score of the mice was assessed as described in Example 6, at day 5 or day 6. The results are shown in FIG. 13. It is observed that the MG1363 bacteria have no effect on DNBS-induced colitis.

### 7.3/ Evaluation of Mn1 Loaded MG1363 Bacteria Towards DNBS Induced Colitis

The first day of the assay (DO), mice were anesthetized with Ketamine and Xylazine 0.06% by intraperitoneal injection and DNBS (3 mg/mouse) and a DNBS solution (Sigma) in 30% ethanol/PBS) was administrated by an intrarectal injection using a catheter at about 3.5 cm into the colon. One group, non-colitis control, received only Hepes and is called “Vehicle”. Bacteria (10⁹ bacteria/200 μL), loaded bacteria (CMB) or Hepes were daily intragastrically administrated to mice for 5 days. The amount of CMB given to the mice was determined in such a way that all mice receive the same amount of total manganese, fixed at 150 nmol per dose. Mice were supervised all along the experiment with special attention.

The weight of all mice was monitored over time. The mice weights at day 5 are shown in FIG. 14. These results demonstrate that administration, to mice with DNBS induced colitis, of MG1363 bacteria loaded with Mn1 increases the weight of the mice, which denotes an improvement in the health of the mice. This improvement is not obtained with the empty bacteria.

### 8/ Statistical Analysis (Example 6 and Example 7)

Statistical analysis was completed using GraphPad Prism software (GraphPad Software). Results are presented as dot plots with means±SEM. The p-values were calculated using the non-parametric Mann-Whitney test (one-tailed test). Outliers found using the Prism software were discarded. The mean ranks of each column were compared to that of the DNBS Hepes control; each comparison stands alone. ****: p<0.0001, ***: p<0.001, **: p<0.01, and *: p<0.05 versus DNBS Hepes control or vehicle, and ns means non-significant. A p value of less than 0.05 was considered significant.

What is claimed is:

1. A method of treating a subject, comprising administering to said subject, as a medicament or a dietary supplement, an effective amount of a chemically-modified microorganism selected from the group consisting of bacteria and yeasts, said chemically-modified microorganism containing a synthetic superoxide dismutase mimic inorganic complex.

2. The method according to claim 1, wherein said inorganic complex is a mimic of human manganese superoxide dismutase.

3. The method according to claim 1, wherein the metal of said inorganic complex is manganese (II) or manganese (III).

4. The method according to claim 1, wherein said inorganic complex has the general formula (I): 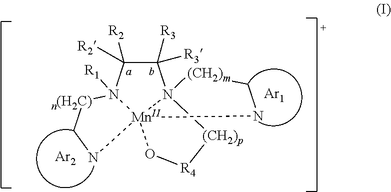
wherein
n, m and p, identical or different, are integers between 1 and 3,
R₁ represents a hydrogen atom or a linear, branched and/or cyclic alkyl, optionally substituted by at least one aromatic cycle or polycycle,
or R₁ represents a peptide comprising from 1 to 20 amino acid residues,
R₂, R₂′, R₃, R₃′ all represent a hydrogen atom,
or R₂′ and R₃′ represent a hydrogen atom and R₂ and R₃ form, together with the carbon atoms to which they are attached, an aliphatic cycle or heterocycle, optionally substituted by a Lewis base, a phenol group, an imidazole ring, a pyridine ring and/or by one or several identical or different alkyl group(s), each of said alkyl group(s) being optionally substituted by a Lewis base, a phenol group, an imidazole ring and/or a pyridine ring,
or R₂, R₂′, R₃, R₃′ form, together with the carbon atoms to which they are attached, an aromatic cycle or heterocycle, optionally substituted by a Lewis base, a phenol group, an imidazole ring, a pyridine ring and/or by one or several identical or different alkyl group(s), each of said alkyl group(s) being optionally substituted by a Lewis base, a phenol group, an imidazole ring and/or a pyridine ring,
Ar₁ and Ar₂, which may be identical or different, each represents a 5-membered or 6-membered aromatic heterocycle, optionally substituted by one or several identical or different substituents, each of which is selected from the group consisting of a bromine atom, a chloride atom, a methyl group, a methoxy group and a nitro group,
R₄ represents a phenyl group, optionally substituted by one or several identical or different substituents, each of which is selected from the group consisting of a bromine atom, a chloride atom, a methyl group, a methoxy group and a nitro group,
or R₄ represents a group of formula —R₅—CO—, wherein the carbonyl group is attached to the oxygen atom and R₅ is attached to the —(CH₂)ₚ— group, and R₅ represents a linear, branched and/or cyclic alkyl, optionally substituted by a Lewis base.

5. The method according to claim 4, wherein said inorganic complex has the general formula (IV): 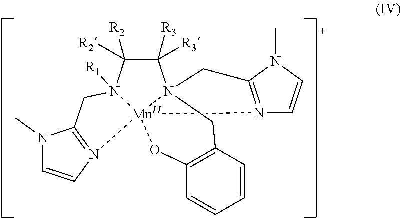
wherein R₁, R₂, R₂′, R₃ and R₃′ are as defined in claim 4.

6. The method according to claim 5, wherein said inorganic complex has the formula (VII):

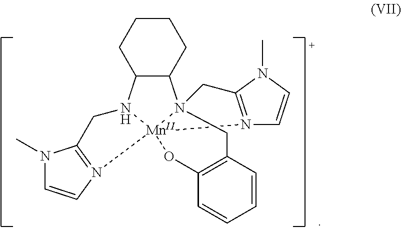

7. The method according to claim 1, wherein said microorganism is a lactic acid bacterium.

8. The method according to claim 1, wherein the cell wall of said microorganism has been genetically, chemically and/or enzymatically weakened.

9. The method according to claim 8, wherein said microorganism is auxotrophic for an amino acid and has been cultivated in a culture medium devoid of said amino acid for at least 1 hour.

10. The method according to claim 1, for combating oxidative stress.

11. The method according to claim 1, for treating an inflammatory disease.

12. The method according to claim 11, for treating an inflammatory bowel disease.

13. The method according to claim 1, wherein said chemically-modified microorganism is administered orally to a said subject.

14. The method according to claim 1, wherein said subject is a mammal.

15. A pharmaceutical composition containing a chemically-modified microorganism as defined in claim 1 in a pharmaceutically suitable vehicle.

16. A nutraceutical composition, food composition or dietary supplement containing a chemically-modified microorganism as defined in claim 1 in a physiologically acceptable vehicle.

17. The method according to claim 7, wherein said microorganism is a lactic acid bacterium of the genus Lactobacillus.

18. The method according to claim 7, wherein said microorganism is a lactic acid bacterium of the genus Lactococcus.

19. The method according to claim 14, wherein said subject is a human.


SEQUENCE LISTING (2 sequences)

SEQ ID NO: 1 - DNA, 20 bp, synthetic construct
  source 1..20: mol_type = other DNA
attaacgcgcagatcatgca

SEQ ID NO: 2 - DNA, 20 bp, synthetic construct
  source 1..20: mol_type = other DNA
tgtcccccaccattgaactt